(19) United States
(12) Patent Application Publication
(10) Pub. No.: US 2002/0169345 A1
(43) Pub. Date: Nov. 14, 2002

(54) Methods and systems for extracting gases

(75) Inventor: Keith H. Johnson, Cambridge, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Supercritical Combustion Corporation, Woburn, MA

(21) Appl. No.: 09/853,838

(22) Filed: May 11, 2001

(51) Int. Cl. C07C 7/00; B01D 7/00

(52) U.S. Cl. 585/15; 422/255

(57) ABSTRACT

This invention relates to methods and systems for extracting hydrocarbon gases from hydrates, and more particularly relates to methods and systems for maximizing the efficiency of energy transfer of electromagnetic (EM) radiation of specified frequencies to the hydrate mass and the dissociation of methane and other trapped hydrocarbon gases from their hydrate cages through a unique EM-induced collective molecular vibronic process.

### BACKGROUND OF THE INVENTION

This invention relates to methods and systems for extracting gases, and more particularly relates to methods and systems for extracting methane and other trapped gas from their hydrate cages.

Gas hydrates are crystalline combinations of one or more gases such as methane, natural gas and other hydrocarbon gas molecules of small linear dimension (i.e., C₁-C₄ or larger carbon containing molecules which have a maximum linear dimension of about 100 nanometers (10 Angstroms) such as neopentane) and water formed into a substance that looks like ice but it is unstable at standard temperature and pressure. The gas hydrate also may contain other light gases (CO₂, H₂S, N₂, etc.). The gas molecules are physically entrapped or engaged in the expanded lattice of the water network comprising hydrogen bonded molecules. The structure is stable due to weak van der Waals' bonding between the gas and the water molecules and hydrogen-bonding between water molecules within the cage structure.

Gas hydrates are found under the ocean floor and in permafrost. Gas hydrate until recently has been considered only a nuisance that can plug up pipelines, but now is viewed by some scientists as the resource that may power the twenty-first century.

Gas hydrates occur abundantly in nature, both in Arctic regions and in marine sediments. They look very much like water ice. Methane hydrate is stable in ocean floor sediments at water depths greater than 300 meters and, where it occurs, it is known to cement loose sediments in a surface layer several hundred meters thick.

It is possible that natural gas can be produced economically from the methane and other gas hydrates on a global scale. The U.S. Geological Survey (USGS) estimates that the methane hydrates beneath U.S. waters alone hold some 200 trillion cubic feet of natural gas, enough to supply all the nation's energy needs for more than 2,000 years at current rates of use. These immense amounts of natural gas have major implications for energy resources and climate, but the natural controls on hydrates and their impacts on the environment are very poorly understood.

The presence of gas hydrates has several hazardous implications and environmental concerns. In the Gulf of Mexico, oil companies are drilling into water more than 1,000 meters deep and are starting to drill through layers of methane hydrate. This can cause the hydrate to dissociate. If the focus is limited to extracting the oil beneath the gas hydrate layers and/or appropriate precautions are not taken, gas may be released which can explode and cause drilling crews to lose control of their wells. Engineers are worried that unstable hydrate layers could give way beneath oil platforms or even play a role in triggering tsunamis. There is also concern that global warming could melt some shallow methane deposits, releasing millions of tons of this potent greenhouse gas into the air.

Gas hydrates have not been economical to harvest for various reasons. Supplying heat as steam at the bottom of a drill hole would be very inefficient because of heat losses to the wall of the hole. Supplying electrical heat would be very inefficient because transmission of that heat to the water-hydrate interface would require a large over-temperature in the area of the heater. Physically mining the deposits and releasing/capturing the gas at the surface would be technically possible but may be economically prohibitive.

Experts say it is hard to know if gas hydrates ever will become a source of fuel, since methods for recovery are not currently available. Even so, the days of plentiful oil and gas are numbered, and countries will require new energy sources to keep their economies moving. Gas hydrates could be that source.

The worldwide amounts of carbon bound in gas hydrates is conservatively estimated to total twice the amount of carbon to be found in all other known fossil fuels on Earth. This estimate is made with minimal information from U.S. Geological Survey (USGS) and other studies. Extraction of methane from hydrates could provide an enormous energy and petroleum feedstock resource. Additionally, conventional gas resources appear to be trapped beneath methane hydrate layers in ocean sediments.

Research efforts have been directed to reducing the problems of gas hydrates in petroleum product lines, by either inhibiting the formation thereof or dissociating gas hydrates which are present and/or recovering the gases from the hydrates for beneficial use. For example, PCT/US97/24292, published Jul. 9, 1998, reviews a variety of prior art research focused upon these. This reference specifically teaches the use of electromagnetic radiation over a broad frequency range 100 megahertz (MHz) to 3000 gigahertz (GHz) (wavelength range 0.1 millimeter (mm) to 3 meter (m)) to heat and dissociate hydrocarbon gas hydrates, with microwave radiation stated as being preferred. Microwave radiation, which has a wavelength in the 0.1 mm to 1 mm range, is widely used to transfer energy to materials containing liquid water (e.g., as in a conventional microwave oven wherein food is heated by the resultant heating of the aqueous component of the food). In the case of a hydrate, sufficient microwave exposure could similarly impart energy to the water molecules and cause breaking of the hydrogen bonds of the water in the clathrate structure as well as overall heating of the water molecules.

### SUMMARY OF THE INVENTION

The present invention methods and systems for transferring energy of a targeted wavelength which efficiently releases the contained gases by electromagnetically inducing collective vibrational modes of the gas hydrate. In one embodiment, a method is provided for selectively releasing and ultimately harvesting the otherwise trapped gas molecules from their hydrate cages. Submillimeter wavelength radiation of 0.1 mm (which is equal to a frequency of 3000 GHz) to 1 mm (which is equal to a frequency of 300 GHz) region of the electromagnetic spectrum is applied to gas and the released gas is collected.

More specifically, submillimeter radiation in the wavelength 0.1 to 1 mm (3000 to 300 GHz frequency) region of the electromagnetic spectrum is applied to methane or other trapped hydrocarbon gas hydrate to excite the large-amplitude gas-hydrate vibrations (i.e., instead of simply imparting heat energy directly to the water molecules) and directly impacting the hydrogen-bonding between the water molecules in the hydrate, the vibrations induced by the targeted electromagnetic radiation of this invention cause the energy gap between the highest-energy occupied gas-hydrate bonding molecular orbitals (HOMOs) and lowest-energy, otherwise unoccupied gas-hydrate antibonding molecular orbitals (LUMOs) to close, pouring electrons from the bonding into the antibonding gas-hydrate orbitals and thereby causing the release of the gas from its water-clathrate cages. This vibronic process much more efficiently releases the gases from the clathrate structure than broad frequency microwave or other electromagnetic heating process.

A component of the applied radiation maybe outside the submillimeter wavelength of 0.1 mm to 1 mm (3000 to 300 GHz frequency) region of the electromagnetic spectrum, but it is preferred that the radiation applied be optimized such that a preponderance of the radiation generated is in this wavelength range, and such optimized radiation most preferably is applied continuously until substantially all methane or other trapped hydrocarbon gas in the targeted area has been recovered.

The gas hydrate to which the radiation is applied is preferably present in situ (e.g., regions in the ocean floor or in permafrost at the depths where the gas hydrate is found). Hydrates contained in storage vessels or in pipelines are also preferred. In the case of recovery of in situ gas hydrates, the submillimeter radiation preferably is applied to regions in which the gas hydrate is in relatively pure or concentrated aggregates and is not surrounded by layers impermeable to the radiation and/or the released gas, which would prevent production from the same drill hole.

In another embodiment of this invention, a system is provided for the extracting gas, preferably methane, from hydrates which includes a radiation generator capable of generating submillimeter wavelength 0.1 mm to 1 mm (3000 to 300 GHz frequency) region of the electromagnetic spectrum and recovery means for capturing the gas released from the hydrate to which the radiation was applied.

Radiation generators are capable of generating radiation in a suitable frequency range of the electromagnetic spectrum. Most preferably, in the practice of this invention the generator produces radiation in the specified narrow band of frequencies or can be tuned to a narrow adsorption band. Preferably, the radiation generator is an array of submillimeter-wavelength small-dish antennae connected remotely via wave guides to gyrotrons emitting a frequency in the submillimeter wavelength 0.1 mm to 1 mm (3000 to 300 GHz frequency) range. The submillimeter radiation generator also may be a free-electron laser.

The released gas may be captured by any gas capture means, such as those used in natural gas exploration and production, including those methods taught in PCT/US97/24292, the teachings of which are incorporated herein by reference, for recovery from storage zones, pipelines and petroleum-bearing rock formations in the vicinity of a production well. The means selected must be suited to the conditions and circumstances in which the targeted gas hydrate exists. For example, in the recovery of methane from methane hydrate deposit on the ocean floor, one would preferably use a capture cone (see FIG. 6). Most preferably, the means selected would capture all or essentially all of the released methane for both economic and environmental reasons.

In order to gather or collect methane or other hydrocarbon gas released from in situ gas hydrate from layers of hydrate capped by sediment, an array of submillimeter-wavelength small-dish antennae connected remotely to gyrotrons via wave guides would be lowered through drilled holes (of sufficient diameter to contain the antennae and produce the gas) (see FIG. 7). The produced gas would be piped upward to the ocean surface into a suitable collection or distribution system (e.g., a pipeline). Other suitable recovery means would be application specific and would be similar to devices used to recover natural gas from under sea strata.

### BRIEF DESCRIPTION OF THE DRAWINGS

FIG. 1 depicts a methane hydrate crystal in its simplest form.

FIGS. 2A, 2B and 2C depict the three structures of methane hydrates.

FIGS. 3A-3F depict orbital structures of hydrate cages.

FIG. 4 depicts the large-amplitude, 300 to 3000 GHz frequency oscillations of the methane molecule resonant with water-cluster cage vibrations.

FIG. 5 depicts closing of the energy gap between the highest-energy occupied methane-hydrate bonding molecular orbitals (HOMOs) and lowest-energy, otherwise unoccupied methane-hydrate antibonding molecular orbitals (LUMOs).

FIGS. 6A and 6B depict a system provided for the recovering methane from methane hydrates on the ocean surface, which includes a radiation generator and recovery means for capturing the released methane.

FIG. 7 depicts a system provided for the recovering methane from methane hydrates beneath sediments which includes a radiation generator and recovery means for capturing the released methane.

### DETAILED DESCRIPTION OF THE INVENTION

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention. Methane hydrates form when water molecules bond with methane molecules.

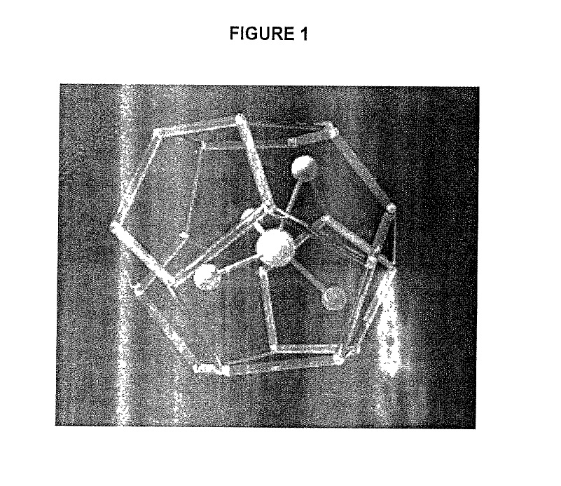

Both water and methane contain hydrogen. One methane molecule contains one carbon atom and four hydrogen atoms (CH₄); one water molecule contains one oxygen atom and two hydrogen atoms (H₂O). Methane hydrates are a form of “clathrate,” which are compounds formed when molecules of one type form a lattice structure around a cavity and molecules of another type are included in the cavity. In its simplest form, a methane hydrate crystal consists of methane molecules surrounded by cages of water molecules as shown in FIG. 1.

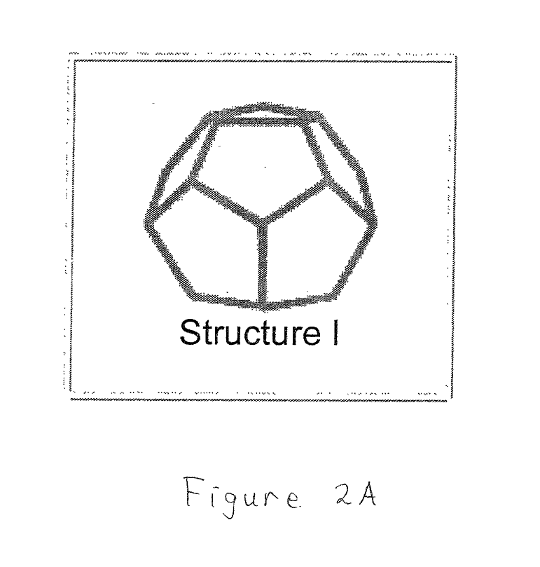

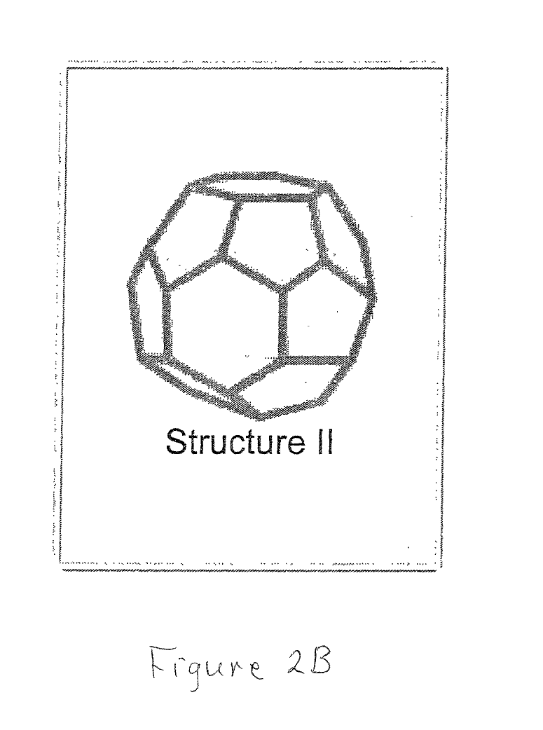

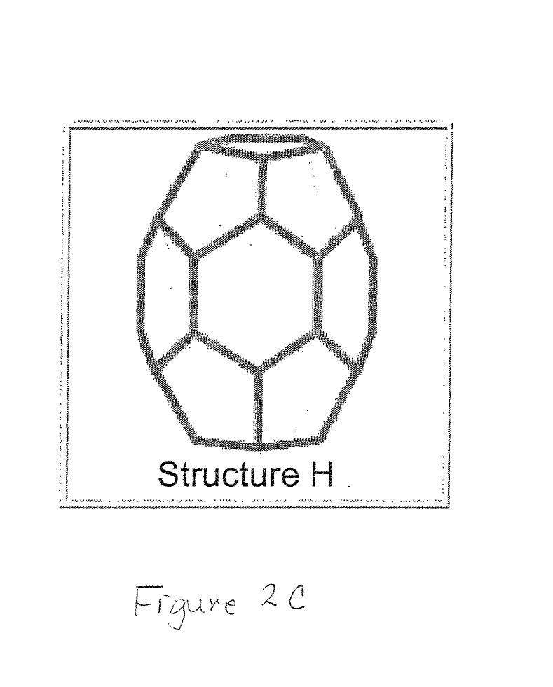

There are three structures of hydrates: I, II, and H, as shown in FIGS. 2A-2C. Each structure has different numbers of water and gas molecules. The ratio of water molecules to gas molecules is called the “hydrate number.” The amount of gas actually contained in a hydrate is called “the degree of filling.”

Structure I hydrates contain 46 water molecules per 8 gas molecules. The hydrate number is 5.75. The water molecules form two small pentagonal dodecahedral voids and six large tetradecahedral voids. These voids can hold only small gas molecules (methane, ethane) with molecular diameters not exceeding 5.2 angstroms.

Structure II hydrates contain 136 water molecules per 24 gas molecules. The hydrate number is 5.67. The water molecules form 16 small dodecahedral voids and 8 large hexakaidecahedral voids. They may contain gases with molecular dimensions from 5.9 to 6.9, such as propane, a three-carbon hydrocarbon, and isobutane. Structure II hydrate was first produced in laboratory experiments. It was first found in a natural environment in 1983 at a depth of 530 meters.

Structure H hydrates contain 34 water molecules per 6 gas molecules. The hydrate number is 5.67. This structure is large enough to hold molecules like isopentane, a branched-chain hydrocarbon molecule with five carbon atoms. Structure H was first found in nature in 1993, at a similar water depth to Structure II, near Jolliet Field, a large oil and gas producing area in the Gulf of Mexico.

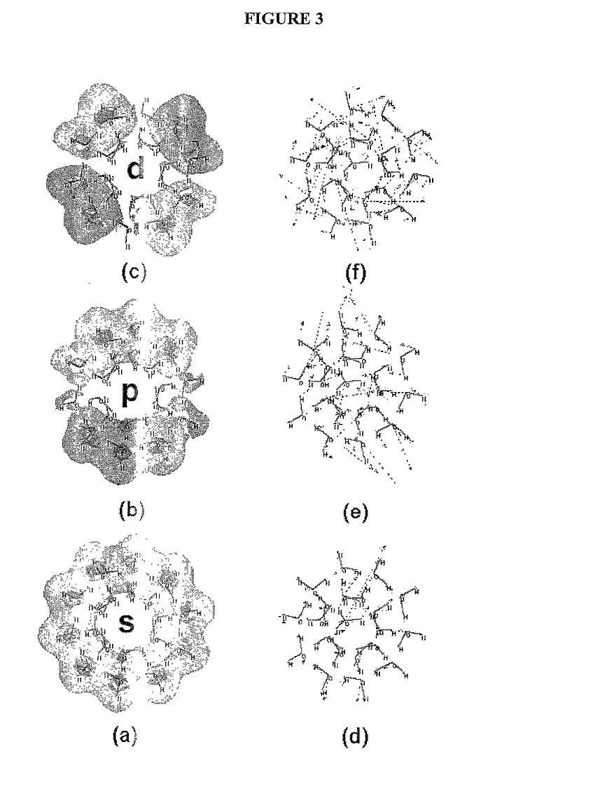

In U.S. Pat. Nos. 5,800,576 and 5,997,590, the teachings of which are incorporated herein by reference, it is taught that the highest-energy molecular electron orbitals of a pentagonal dodecahedral water cluster of the type forming the Structure 1 hydrate cage (FIGS. 1 and 2) are in the form of gigantic “S,” “p,” and “d” orbitals (FIGS. 3A-3C). This unique electronic structure of a water clathrate cage gives rise to 300 to 3000 GHz frequency (submillimeter wavelength) “Hg squashing” vibrational modes of the type shown in FIGS. 3D-3F, where the vibrational amplitudes are represented by arrows.

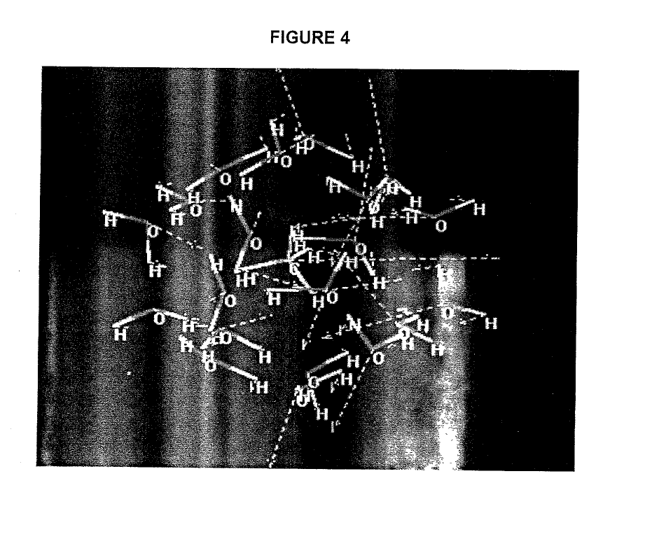

While not intending to be bound by the mechanism involved, it is believed that methane molecules clathrated in a dodecahedral water-molecule cage like that shown in FIGS. 1, 2A-2C and 3A-3F interact with the cage electronically via the overlapping water-cluster molecular orbitals (FIGS. 3A-3C) and vibrationally via the 300 to 3000 GHz frequency water-cluster “Hg squashing” vibrational modes shown in FIGS. 3D-3F. The resulting vibronic coupling between each clathrated methane molecule within its water-cluster cage can produce large-amplitude, 300 to 3000 GHz frequency oscillations of the methane molecule resonant with the water-cluster cage vibrations, as shown in the FIG. 4 resulting from ab initio quantum-chemistry computations. Similar results are obtained for methane hydrate structures II and H in FIGS. 2B and 2C, as well as for other hydrocarbon (e.g. neopentane) gas hydrates of small linear dimension. It should be emphasized that the vibrations of a free methane molecule or other hydrocarbon gas molecule are confined to much higher-frequency C-H “stretching” modes, so the vibronic property is unique to gas hydrates.

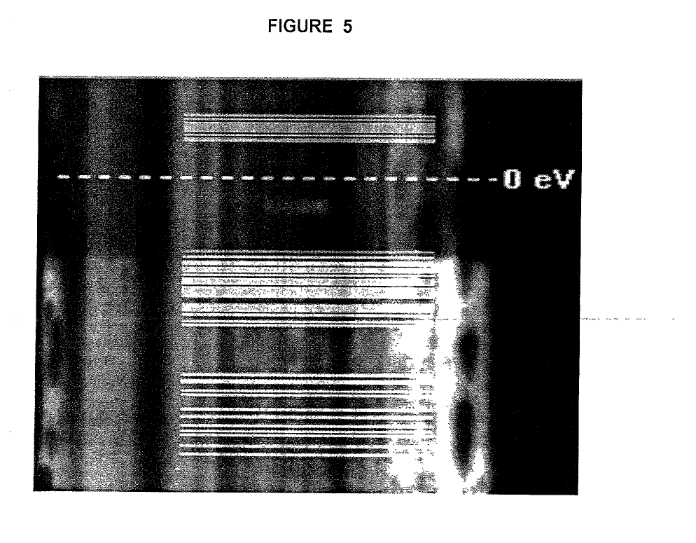

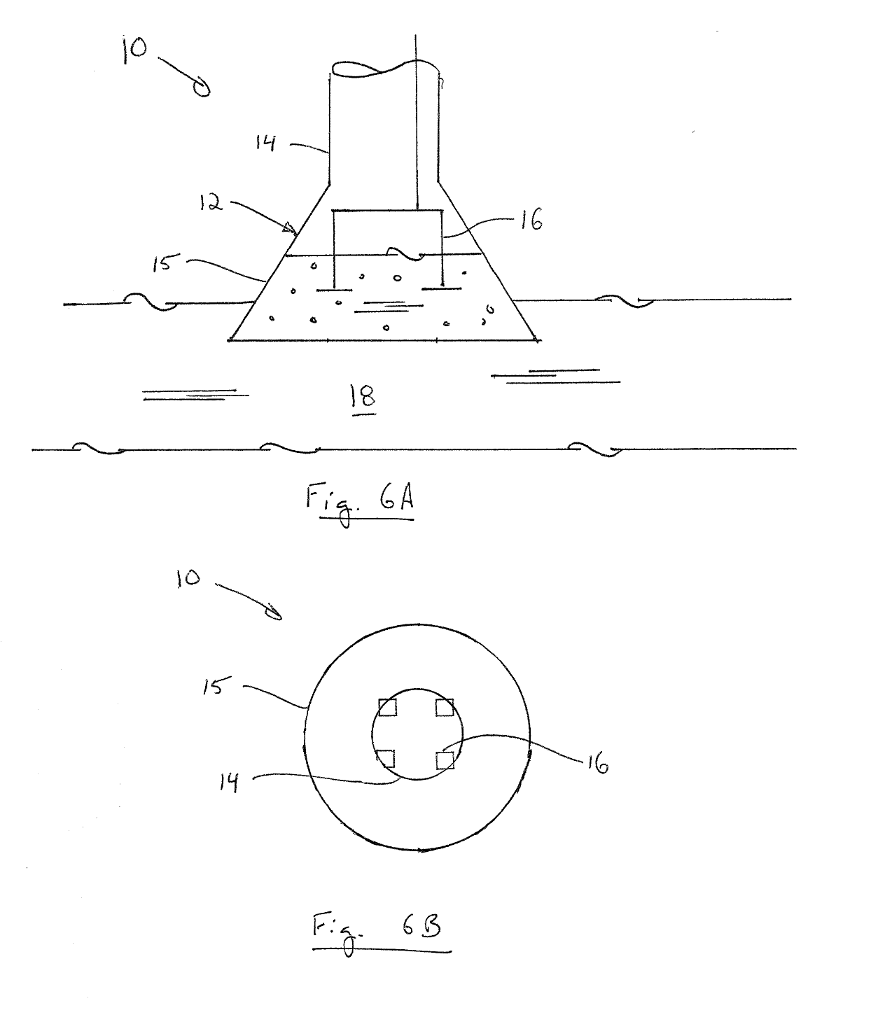

Submillimeter radiation optimized to the 300 to 3000 GHz frequency region of the electromagnetic spectrum is applied to methane hydrate to excite the preferred large-amplitude methane-hydrate vibrations. Such vibrations cause the energy gap between the highest-energy occupied methane-hydrate bonding molecular orbitals (HOMOs) and lowest-energy, otherwise unoccupied methane-hydrate antibonding molecular orbitals (LUMOs), shown in FIG. 5, to close, pouring electrons from the bonding into the antibonding methane-hydrate orbitals and thereby causing the release of methane from its water-clathrate cages.

Generally, the method of the invention includes an electromagnetic wave-induced molecular vibronic process for recovering gas from gas hydrates, comprising exposing gas hydrate to radiation in the wavelength of between about 0.1 and about 1 mm (between about 3000 and about 300 GHz frequency) of the electromagnetic spectrum, resulting in the release of gas molecules and recovering the released gas. In one embodiment, the hydrate is present beneath the ocean floor and exposure to the electromagnetic radiation occurs in situ. In another embodiment, the gas hydrate is in permafrost and, again, exposure to electromagnetic radiation by the method of the invention occurs in situ. Alternatively, the gas hydrate that is exposed to electromagnetic radiation by the method of the invention can be in a storage zone or a gas pipeline.

The gas contained in the gas hydrate can be selected from the group, for example, of methane, natural gas, and other hydrocarbon gas molecules of small linear dimension. In one specific embodiment, the gases contained in the gas hydrate are hydrocarbon gas molecules of small linear dimension together with other light gases.

In another embodiment, the invention is a system for recovering gas from a gas hydrate, comprising a generator for producing radiation having a wavelength in a range of between about 0.1 mm and about 1 mm (between about 3000 GHz and about 300 GHz), and a recovery means for capturing gas released from the gas hydrate which has been exposed to the radiation. In one embodiment, the generator is a free electron laser. In another embodiment, the radiation generator is an array of submillimeter-wavelength small-dish antennae connected remotely via wave guides to gyrotrons. The recovery means can be, for example, a capture cone. In one specific embodiment, the generator produces radiation that predominantly has a wavelength in a range of between about 0.1 mm and about 1 mm (between about 3000 GHz and about 300 GHz frequency region of the electromagnetic spectrum).

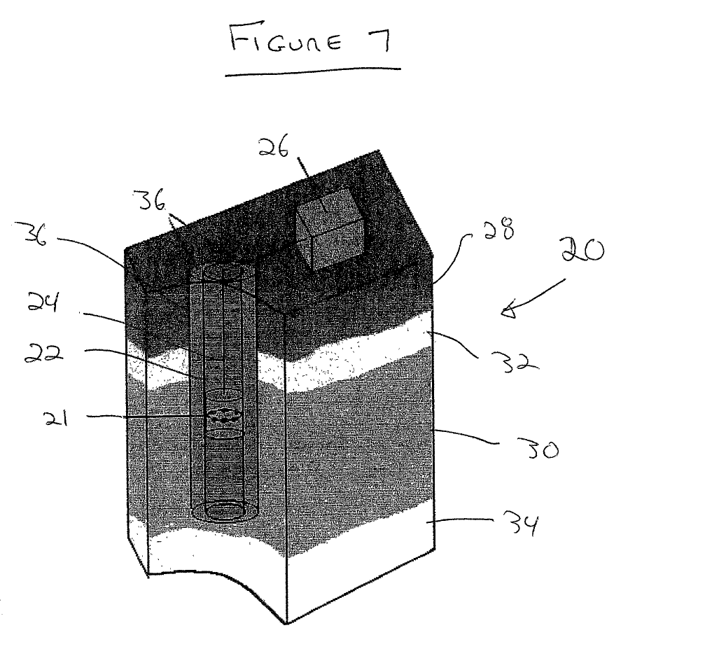

FIGS. 6A and 6B are side and plan views, respectively, of a system of the invention for recovering gas from gas hydrate. As shown therein, system 10 includes radiation generator 12. Radiation generator 12 includes power/signal conduit 14, capture cone 15 and terahertz signal radiator 16. When in use, radiation generator 12 rests on or within hydrate deposit 18. Activation of system 10 generates electromagnetic waves in a wavelength between about 0.1 to 1 mm (3000 to 300 GHz frequency) and consequent release of gas molecules from hydrate deposit 18 by the method of the invention. The released gas is collected and transported through capture cone 15 and power/signal conduit 16 by suitable means to a collection vessel, not shown.

FIG. 7 is a three-dimensional representation of a system of the invention in a typical application. As shown in FIG. 7, system 20 includes terahertz signal radiator 21. Terahertz signal radiator 21 is suspended within well 22 by cable 24. Cable 24 connects terahertz signal radiator 21 to terahertz transmitter electronics 26, which can be located on, for example, an ocean or permafrost surface 28. Terahertz signal radiator 21 is located within gas hydrate layer 30. Typically, gas hydrate layer is located between sediment layers 32 and 34. During use, terahertz signal radiator denerates electromagnetic radiation at a wavelength in a range of between about 0.1 and about 1 mm (between about 3000 and about 300 GHz frequency). The gas that is released from a hydrate state by use of the terahertz signal radiator 20 is conducted to ocean or permafrost surface 28 through conduit 36 at the perimeter of well 22.

Exemplification

A methane hydrate is formed as follows: To a 200 cc pressure vessel is added 100 g water. The vessel is jacketed and has a transducer capable of emitting an electromagnetic radiation predominately of 0.2 mm wavelength (a frequency of 1,500 GHz) and a thermocouple mounted in the bottom. The water is cooled to 0° C., thereby freezing it. A vacuum is drawn. The system is sealed off and the water melted. Vacuum is drawn again. The water is heated to 10° C. To the gas connection is added 20.96 g of methane (29.33 L, at Standard Temperature and Pressure (STP)). As the first 5.27 g is added, the pressure rises to 1110 psig and stabilizes there while the rest of the methane is added. This indicates that hydrate formation has taken place.

Methane then is released from the methane hydrate through the practice of this invention, as follows: the transducer is activated with 2 watts, energy and emits an electromagnetic radiation predominately of a 0.6 mm wavelength. The temperature of the system rises to 12° C. Methane gas is allowed to bleed off, maintaining a pressure of 1400 psig. The system stays at 12° C. until no more methane is given off. The methane is collected in a series of inverted 10 L graduates. After 30 minutes, 19.5 L of methane is recovered in the graduates. This indicates almost complete methane recovery.

As a counter or control experiment, the same experiment is carried out on the same amount of methane hydrate, except that the transducer, activated with the same 2 watts of energy, is adjusted to emit a frequency of 1 GHz (300 mm wavelength electromagnetic radiation). The water heats only very slowly and never reaches 12° C. because of the cooling of the jacket. The pressure in the system rises only a few pounds to about 1125 psig, indicating that little or no methane was released in 30 minutes of operation.

Equivalents

While this invention has been particularly shown and described with references to preferred embodiments thereof, it will be understood by those skilled in the art that various changes in form and details may be made therein without departing from the scope of the invention encompassed by the appended claims.

We claim:

1. An electromagnetic-wave-induced molecular vibronic process for recovering gas from gas hydrates comprising: exposing the gas hydrate to radiation in the wavelength 0.1 to 1 mm, resulting in the release of gas molecules and recovering the released gas.

2. The process of claim 1, wherein said hydrate is present beneath the ocean floor and said exposure occurs in situ.

3. The process of claim 1, wherein said gas hydrate is in permafrost and said exposure occurs in situ.

4. The process of claim 1, wherein said gas hydrate is in a storage zone.

5. The process of claim 1, wherein said gas hydrate is in a pipeline.

6. The process of claim 1, wherein the gas contained in said gas hydrate is selected from the group consisting of methane, natural gas and other hydrocarbon gas molecules of small linear dimension.

7. The process of claim 1, wherein the gases contained in said gas hydrate are hydrocarbon gas molecules of small linear dimension together with other light gases.

8. A system for recovering gas from a gas hydrate comprising: a generator producing radiation having a wavelength in a range of between about 0.1 and about 1 mm, and recovery means for capturing gas released from the gas hydrate which has been exposed to said radiation.

9. The system of claim 8, wherein said generator is a free electron laser.

10. The system of claim 8, wherein said radiation generator is an array of submillimeter-wavelength small-dish antennae connected remotely via wave guides to gyrotrons.

11. The system of claim 8, wherein said recovery means is a capture cone.

12. The system of claim 8, wherein said generator produces radiation which is predominately in the 0.1 to 1 mm.
